(19) United States
(12) Patent Application Publication
(10) Pub. No.: US 2008/0208204 A1
(43) Pub. Date: Aug. 28, 2008

(54) INTRAARTICULAR GRAFT LENGTH GAUGE

(75) Inventors: Reinhold Schmieding, Naples, FL (US); Jacob Jolly, Naples, FL (US); Ricardo Albertorio, Naples, FL (US); Gregory Joshua Karnes, Naples, FL (US)

Correspondence Address: DICKSTEIN SHAPIRO LLP, 1825 EYE STREET NW, Washington, DC, 20006-5403, US

(73) Assignee: Arthrex, Inc.

(21) Appl. No.: 12/030,773

(22) Filed: Feb. 13, 2008

Related U.S. Application Data
(60) Provisional application No. 60/900,988, filed on Feb. 13, 2007.

(51) Int. Cl. A61B 17/90

(52) U.S. Cl. 606/102

(57) ABSTRACT

A technique and reconstruction system for ligament repair by determining the exact length of the graft to be secured within a first tunnel or socket (for example, a tibial tunnel) and a second tunnel or socket (for example, a femoral tunnel) for improved graft fixation. The exact length of the graft (soft tissue graft or BTB graft, for example) is determined with a measuring device, by measuring the length of a first socket in a first bone region (for example, a tibial socket), the length of a second socket in a second bone region (for example, a femoral socket), and the length of the intraarticular joint space between the two sockets in the two bone regions (for example, tibia and femur). The measuring device may consist of at least one flexible member (for example, suture) with at least one fixation device attached to the flexible member, the fixation device being either a sliding knot, a washer or a button.

### CROSS REFERENCE TO RELATED APPLICATIONS

This application claims the benefit of U.S. Provisional Application Ser. No. 60/900,988 filed on Feb. 13, 2007, the entire disclosure of which is incorporated by reference in its entirety herein.

### FIELD OF THE INVENTION

The present invention relates to the field of surgery and, more particularly, to a method and apparatus for improved graft fixation in ACL reconstructive surgeries.

### BACKGROUND OF THE INVENTION

Reconstructive surgeries, particularly anterior cruciate ligament (ACL) reconstruction, are well-known in the art. In general, these methods of tenodesis involve drilling a tunnel through the tibia, drilling a closed tunnel (socket) into the femur, inserting a substitute ACL graft into the tunnels, and securing the grafts to the walls of the tibial and femoral tunnels using interference screws or the like.

Recently, an “all-inside” ACL technique has been developed, wherein two closed tunnels (sockets) are drilled, one through the tibia and one through the femur. The femoral socket is formed by using a retrograde drill device provided with a retrograde drill cutter detachable from a retrograde drill guide pin, in the manner described in U.S. Patent Application Publication No. 2004/0199166, the disclosure of which is hereby incorporated by reference herein in its entirety. The tibial tunnel or socket may be formed by the retrograde drill method or by a conventional method, and may be carried out before or after the formation of the femoral socket.

Graft length determination in the above-described “all-inside” retrograde drill technique is difficult, as adjustment of the length of the graft (by cutting the graft at the end of the tibial tunnel, for example) is impossible since the tunnels are not drilled from the outer cortex (as in a conventional ACL technique). It is desirable, therefore, to use a graft with a predetermined yet exact length to be secured intraarticularly within the sockets (tunnels). A graft with a predetermined, exact length would eliminate any movement of the graft from side to side (increasing the life of the ACL repair) and would allow proper tensioning of the graft subsequent to its fixation. A method for determining the appropriate length of a graft for positioning of the graft intraarticularly is also desirable.

### BRIEF SUMMARY OF THE INVENTION

The present invention provides a technique and reconstruction system for ligament repair by determining the exact length of the graft to be secured within a first tunnel or socket (for example, a tibial tunnel) and a second tunnel or socket (for example, a femoral tunnel) for improved graft fixation.

The system of the present invention comprises a measuring device for determining the appropriate length of the graft (soft tissue graft or BTB graft, for example) by determining the length of a first socket in a first bone region (for example, a tibial socket), the length of a second socket in a second bone region (for example, a femoral socket), and the length of the intraarticular joint space between the two sockets in the two bone regions (for example, tibia and femur). The measuring device may consist of at least one flexible member (for example, suture) with at least one fixation device attached to the flexible member, the fixation device being selected from the group consisting of a sliding knot, a washer and a button.

Other features and advantages of the present invention will become apparent from the following description of the invention which refers to the accompanying drawings.

### BRIEF DESCRIPTION OF THE DRAWINGS

FIG. 1 illustrates an exemplary suture-button construct for graft length determination according to the present invention.

FIG. 2 illustrates a measuring device comprising a suture and two sliding knots for graft length determination according to the present invention.

FIGS. 3-5 illustrate various steps of a method of ACL reconstruction employing the measuring device of FIG. 2 and according to an exemplary embodiment of the present invention.

FIGS. 6 and 7 illustrate a continuous loop/button construct used for fixating a graft with a length predetermined according to the present invention and used during ACL reconstruction.

### DETAILED DESCRIPTION OF THE INVENTION

The present invention provides a technique and reconstruction system for ligament or tendon repair. The system of the present invention comprises a measuring device designed to determine the appropriate graft length to achieve strong graft fixation and tensioning during ACL reconstruction. The measuring device allows proper tensioning and fixation of the graft in an “all-inside” ACL technique, wherein use of special measurement devices (such as a drill pin provided with laser marks to measure the distance from the femoral notch to the outer cortex, by itself or in conjunction with a depth guide, for example) is not possible due to the way the femoral and tibial tunnels are formed.

The system of the present invention comprises a measuring device for determining the appropriate length of the graft (for example, soft tissue graft or BTB graft) by determining the length of the first socket in a first bone region (for example, the tibial socket), the length of the second socket in a second bone region (for example, the femoral socket), and the length of the intraarticular joint space between the two bone regions (for example, tibia and femur).

According to one embodiment of the present invention, the measuring device is a miniature version of the suture-button construct described in U.S. Appl. Publ. No. 2007/0016208, the entire disclosure of which is incorporated by reference herein. In another embodiment, the measuring device consists of a flexible member (for example, suture) with two sliding knots. In alternative embodiments, the measuring device comprises of a flexible member (for example, suture) with at least one fixation device selected from the group consisting of a sliding knot, a washer and a button.

The invention also provides a method of ACL reconstruction using a graft having a length that is predetermined by measuring, intraarticularly, the length of the tibial and femoral sockets plus the intraarticular space between tibia and femur. The method of ACL reconstruction comprises, for example, the steps of: (i) drilling at least a femoral tunnel or socket and at least a tibial tunnel or socket; (ii) determining the entire length of the sockets plus the intraarticular space by employing a miniature suture-button construct and/or a measuring device consisting of a flexible member (for example, suture) with at least one fixation device selected from the group consisting of a sliding knot, a washer and a button; (iii) determining the length of the graft (soft tissue graft or BTB graft) based on the entire length of the sockets plus the intraarticular space between them; and (iv) securing the graft within the femoral and tibial tunnels (sockets).

According to yet another embodiment, a method of ACL reconstruction of the present invention comprises the steps of: (i) drilling at least a femoral and tibial tunnel or socket using a retrograde drill technique; (ii) determining a first distance D₁ (which is the length of the tibial and femoral sockets plus the intraarticular space) by employing a miniature suture-button construct and/or a measuring device consisting of a flexible member (for example, suture) with at least one fixation device selected from the group consisting of a sliding knot, a washer and a button; (iii) determining a distance D₂ (which is equal to D₁ minus about 5 to about 10 mm); (iv) providing a graft (soft tissue graft or BTB graft) and cutting the graft to a length about equal to D₂; (v) securing the graft (soft tissue graft or BTB graft) to a continuous loop/button construct comprising a button with an oblong configuration and provided with an inside eyelet that allows the passage of the continuous loop, preferably a suture loop; (vi) passing the graft with the button through the femoral tunnel; and (vii) securing the button to the femoral cortex once the button exits the femoral socket.

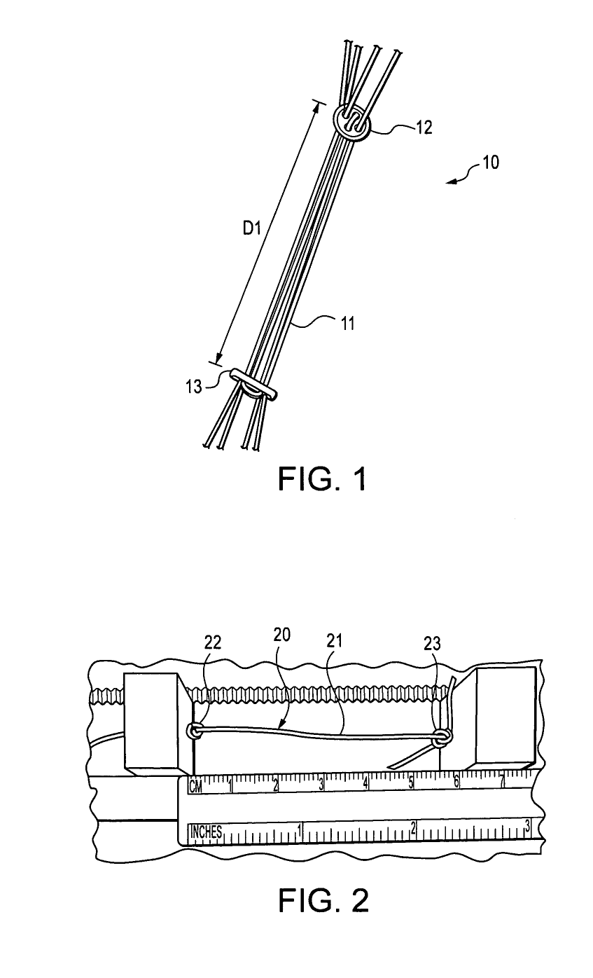

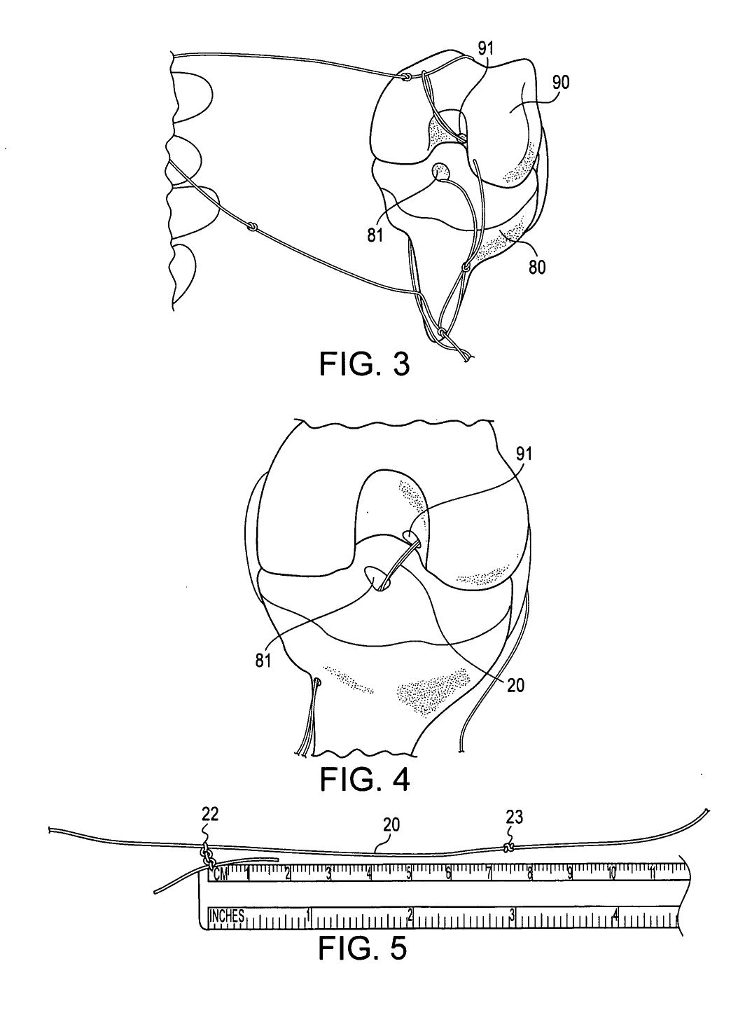

Referring now to the drawings, where like elements are designated by like reference numerals, FIGS. 1 and 2 illustrate exemplary embodiments of measuring devices 10, 20 of the present invention. FIGS. 3-5 illustrate various steps of a method of ACL reconstruction employing the measuring device 20 of FIG. 2 and according to an exemplary embodiment of the present invention.

FIG. 1 illustrates a miniature suture-button construct 10, a miniature version of the device described in U.S. Appl. Publ. No. 2007/0016208. Construct 10 may comprise, for example, two buttons 12, 13 (FIG. 1) each having an oblong body defining first and second apertures, or a button and a washer, among other configurations.

The suture-button construct 10 may comprise a high strength suture, such as Arthrex FiberWire® or lasso wire 11. Exemplary steps for employing the suture-button construct 10 to determine the appropriate graft length are as follows: Two ends of the suture and passed through one button so the loop is at the bottom of the first button. This allows tying of passing sutures to graft and will bottom out the first button at the end of the loop. The free ends of the suture are passed through the eyelets of another round button, loop wire or suture and looped twice, to reduce slippage. Another loop is created on the other end of the suture tails to tie graft passing sutures. The construct is pulled into the joint with the graft passing sutures, bottoming out with the fixed button first (tibia), then the other end is tensioned and bottomed out until the suture or wire between the sockets is tight. The midsection of the gauge (of the suture strand 11) is grabbed through a cannula with a grasper and pulled out until the buttons are in the cannula to avoid slippage on removal through the anteriomedial portal. The distance D₁ between the two buttons 12, 13 may be exactly the length of the graft since the greater diameter of the tendons positioned in the middle of the tunnels may end up giving the 5-10 mm tensioning distance, since the gauge 11 extends from rim to rim versus center to center with a tight fitting graft. Alternatively, the length of the graft may be determined based on the distance D₁ between the two buttons minus about 5 to about 10 mm, to allow tensioning of the graft upon insertion within the bone tunnels/sockets.

Subsequent to the determination of distance D₁, grafts (for example, allografts) may be prepared for fixation using the Arthrex RetroButton™ (continuous suture loop/button construct) technique, for example, and extra long Fiberloop™ stitching, to speed preparation. The construct 10 is positioned on the graft and the final few stitches of the Fiberloop™ removed, to shorten to the exact length needed. In this manner, preparation of the graft with just drilling and measuring is achieved.

Once the length of the graft is adequately determined, preparation of the allograft may be conducted by employing a continuous loop/button construct 30 (FIGS. 6 and 7) provided with a button 33, preferably of titanium alloy, and a continuous loop 35 attached to the button 33. The button 33 may have an oblong configuration and a width that is preferably less than about 1 mm narrower than the width of the drill hole through which the button 33 is inserted and subsequently passed through. The button 33 is provided with an inside eyelet that allows the passage of the continuous loop. In an exemplary embodiment, the suture loop 35 may be a single high strength suture such as FiberWire® suture, sold by Arthrex, Inc. of Naples, Fla., and described in U.S. Pat. No. 6,716,234, the disclosure of which is incorporated by reference herein. In another exemplary embodiment, the continuous loop 35 may be formed of a plurality of suture strands configured to separate from a single strand to a plurality of strands in a continuous loop.

In an exemplary embodiment, the continuous loop/button construct 30 is used to secure a soft tissue graft (measured according to an embodiment of the present invention) in a bone socket in a retrograde manner, for example. According to another exemplary embodiment, the continuous loop/button construct 30 is used to secure a bone-to-bone (BTB) graft (measured according to an embodiment of the present invention) in a femoral tunnel or socket in a retrograde manner, for example.

In these particular and only exemplary embodiments, a method of ACL reconstruction using the continuous loop/button construct and graft (measured according to an embodiment of the present invention) comprises, for example, the steps of: (i) drilling at least one femoral tunnel or socket, and at least one tibial tunnel or socket, wherein the drilling of the at least one femoral tunnel may be conducted using a retrograde drill cutter which is inserted in a retrograde manner through the femur; (ii) determining the entire length of the sockets plus the intraarticular space by employing the miniature suture-button construct 10; (iii) determining the length of the graft (soft tissue graft or BTB graft) based on the entire length of the sockets plus the intraarticular space between them, as measured by the suture-button construct 10; and (iv) securing the graft within the femoral and tibial tunnels (sockets).

The at least one femoral socket may be prepared by employing a retrograde drill device provided with a retrograde drill cutter detachable from a retrograde drill guide pin, in the manner described in U.S. Patent Application Publication No. 2004/0199166. As described in U.S. Patent Application Publication No. 2004/0199166, a retrograde drill device for ACL reconstruction is provided with a retrograde drill cutter detachable from a retrograde drill guide pin. The retrograde drill cutter is inserted in a retrograde manner through the femur by employing a retrograde drill guide pin provided with depth markings. Formation of the tibial tunnel or socket by the method described above or by a conventional method may be carried out before or after the formation of the femoral socket.

Once the femoral and tibial tunnels or sockets have been completed, graft insertion and fixation may be subsequently carried out. According to an exemplary embodiment of the present invention, the graft (which may be a soft tissue graft) is folded in half over the loop of the button 30 and tension is applied. Subsequently, passing sutures are pulled and the graft is passed into the femoral tunnel or socket. When the graft reaches the opening of the femoral socket or tunnel on the femoral cortex, a slight popping sensation may be felt as the button exits and begins to flip horizontally on the femoral cortex. Distal traction on the graft and release of the passing sutures facilitate complete deployment of the button. The passing suture may be removed and tibial fixation may be completed.

The exemplary techniques of ACL reconstruction detailed above are further described below with reference to FIGS. 3-5 and using a measuring device 20 which consists of a flexible member 21 (for example, suture) with two sliding knots 22, 23. In alternative embodiments, the measuring device comprises a flexible member (for example, suture) with at least two fixation devices selected from the group consisting of a sliding knot, a washer and a button. The flexible member may be a suture strand, for example, a single high strength suture such as FiberWire® suture, sold by Arthrex, Inc. of Naples, Fla., and described in U.S. Pat. No. 6,716,234, the disclosure of which is incorporated by reference herein. In additional embodiments, the flexible member may be formed of a plurality of flexible strands, at least one of the strands being a suture strand.

The measuring device 20 is employed to determine the appropriate graft length, in a manner similar to the one described above with reference to the suture-button construct 10. Specifically, an exemplary method of determining the appropriate length of a graft to be secured within a first opening/tunnel within a first bone region and within a second opening/tunnel within a second bone region, comprises the steps of: (i) drilling a femoral tunnel or socket 91 using, for example, a retrograde drill cutter which is inserted in a retrograde manner through femur 90; (ii) drilling a tibial tunnel or socket 81 through tibia 80; (iii) determining the entire length of the sockets 81, 91 plus the length of the intraarticular space by employing the measuring device 20; (iv) determining the length of the graft (soft tissue graft or BTB graft) based on the entire length of the sockets 81, 91 plus the intraarticular space between them, as measured by the device 20; and (v) securing the graft within the femoral and tibial tunnels (sockets) 81, 91.

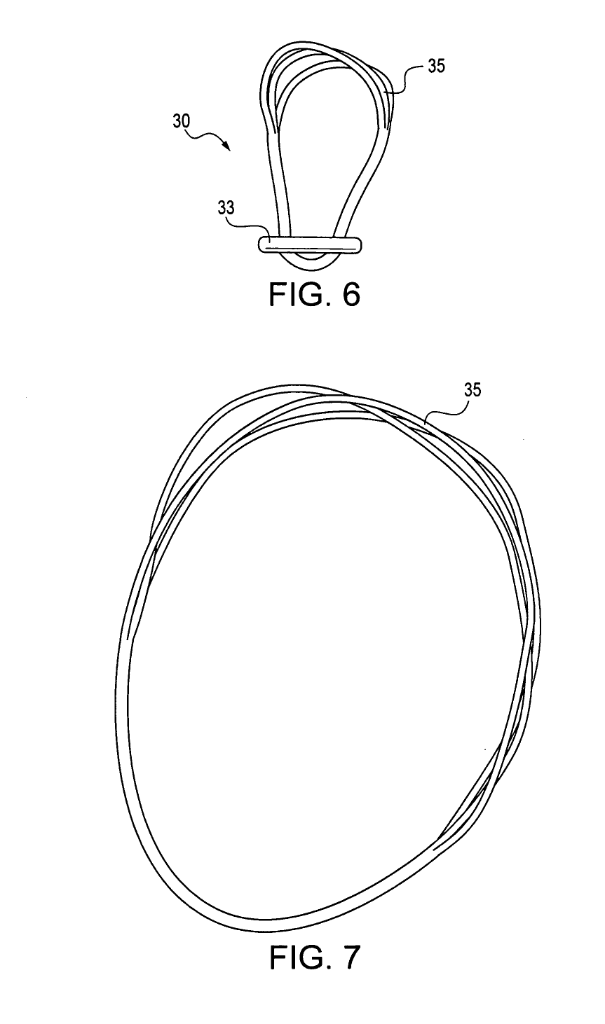

Once the length of the graft is adequately determined, the graft is dimensioned according to the length indicated by the distance between the two sliding knots 22, 23 of the device 20 (FIG. 5), and the dimensioned graft is subsequently prepared for retrograde fixation, for example. A continuous loop/button construct 30 (FIGS. 6 and 7) provided with a button 33, preferably of titanium alloy, and a continuous loop 35 attached to the button 33 may be employed for the graft fixation, and as described above.

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting. Accordingly, it is not intended that the present invention be limited to the illustrated embodiments, but only by the appended claims.

What is claimed as new and desired to be protected by Letters Patent of the United States is:

1. A measuring device for determining the length of a graft to be secured within a first bone tunnel and a second bone tunnel, the measuring device comprising:
at least one flexible strand; and
at least one fixation device affixed to the flexible strand, wherein the at least one fixation device is selected from the group consisting of a knot, a button and a washer.

2. The measuring device of claim 1, wherein the measuring device comprises two fixation devices affixed to the flexible strand.

3. The measuring device of claim 2, wherein the two fixation devices are two sliding knots.

4. The measuring device of claim 2, wherein the two fixation devices are two buttons.

5. The measuring device of claim 1, wherein the at least one flexible strand is a high strength suture.

6. The measuring device of claim 1, wherein the flexible strand has two fixation devices spaced apart from each other by a distance which is about equal to the length of the graft to be secured within the first bone tunnel and within the second bone tunnel.

7. The measuring device of claim 6, wherein the two fixation devices are two sliding knots and the graft is an ACL graft.

8. The measuring device of claim 6, wherein the distance between the two fixation devices is about equal to a sum of a first length of the first bone tunnel, a second length of the second bone tunnel, and a third length of an intraarticular space between the first bone tunnel and the second bone tunnel.

9. A measuring construct for determining the total length of a graft construct to be secured within a femoral tunnel and a tibial tunnel, the measuring construct comprising:
a loop of flexible material having a length about equal to a total length of the graft construct, the length being determined by adding a first length of the femoral tunnel, a second length of the tibial tunnel, and a third length representing the distance between the femoral tunnel and the tibial tunnel;
a first button attached to the loop of flexible material; and
a second button attached to the loop of flexible material.

10. The measuring construct of claim 9, wherein each of the first and second buttons comprises at least one opening configured to allow the loop to pass through it.

11. The measuring construct of claim 9, wherein the loop is formed of a suture material comprising ultrahigh molecular weight polyethylene.

12. The measuring construct of claim 9, wherein at least one of the first and second buttons has an oblong configuration.

13. The measuring construct of claim 9, wherein at least one of the first and second buttons has a length of about 10 mm to about 20 mm.

14. The measuring construct of claim 9, wherein at least one of the first and second buttons has a width that is less than about 1 mm narrower than the width of the femoral tunnel through which the button is inserted and passed through.

15. The measuring construct of claim 9, wherein at least one of the first and second buttons is formed of a material selected from the group consisting of titanium, titanium alloy, polyethylene, PEEK and PLLA.

16. A method of determining the length of tissue to be secured within a first bone tunnel and a second bone tunnel, the method comprising the steps of:
providing a measuring device in the vicinity of the first and second bone tunnels, the measuring device comprising at least one flexible strand and at least one fixation device attached to the flexible strand, the at least one fixation device being selected from the group consisting of a knot, a button and a washer;
measuring, with the measuring device, a total length representing the sum of a first length of the first bone tunnel, of a second length of the second bone tunnel, and of a third length of the intraarticular space between the first bone tunnel and the second bone tunnel; and
dimensioning the tissue based on the total length measured with the measuring device.

17. The method of claim 16, wherein the step of dimensioning the tissue further comprises the step of cutting the tissue to a length which is about equal to the total length measured with the measuring device.

18. The method of claim 16, wherein the step of dimensioning the tissue further comprises the step of cutting the tissue to a length which is about equal to the total length measured with the measuring device minus about 5 to about 10 mm.

19. The method of claim 16, wherein the measuring device comprises at least one suture strand and two sliding knots.

20. The method of claim 16, wherein the measuring device comprises at least one suture strand and two buttons formed of titanium or titanium alloy.

21. The method of claim 16, wherein the tissue to be dimensioned is biological or non-biological tissue.

22. The method of claim 16, wherein the tissue to be dimensioned is selected from the group consisting of ligament, tendon, bone and cartilage.

23. The method of claim 16, wherein the tissue to be dimensioned is soft tissue graft or BTB graft.

24. The method of claim 16, wherein at least one of the first and second bone tunnels is formed in a retrograde manner by using a rotary drill cutter.

25. The method of claim 24, wherein the rotary drill cutter comprises two opposed sides and is provided with cutting surfaces on both sides, such that the rotary drill cutter is configured for cutting in two directions.

26. The method of claim 16, further comprising the steps of:
attaching the dimensioned tissue to a suture loop/button construct having a button with at least one eyelet and a continuous suture loop attached to the at least one eyelet;
pulling the suture loop/button construct with the attached dimensioned tissue through the first and second bone tunnels;
positioning the attached dimensioned tissue within one of the first and second bone tunnels; and
securing the button on a surface of the bone.

27. A method of ligament reconstruction, comprising the steps of:
forming a first socket in a first bone by conducting a first action using a rotary drill cutter, the first bone articulating in a predetermined manner with a second bone;
forming a second socket in the second bone by conducting a second action using the rotary drill cutter;
providing a measuring device in the vicinity of the first and second sockets, the measuring device comprising at least one flexible strand and at least one fixation device attached to the flexible strand, the at least one fixation device being selected from the group consisting of a knot, a button and a washer;
measuring, with the measuring device, a total length representing the sum of a first length of the first socket, of a second length of the second socket, and of a third length of the intraarticular space between the first and second bones;
dimensioning a graft based on the total length measured with the measuring device to obtain a dimensioned graft;
providing a suture loop/button construct in the vicinity of the first and second sockets, the suture loop/button construct comprising a button with at least one eyelet and a continuous suture loop attached to the at least one eyelet;
attaching the dimensioned graft to the suture loop of the suture loop/button construct;
pulling the suture loop/button construct with the attached dimensioned graft through the first socket;
securing the button of the suture loop/button construct to a bone cortex abutting the first socket; and
securing the dimensioned graft within the second socket.

28. The method of claim 27, wherein the first bone is tibia and the second bone is femur.

29. The method of claim 27, wherein the first bone is femur and the second bone is tibia.

30. The method of claim 27, wherein the at least one flexible strand is a suture strand and the at least one fixation device comprises two sliding knots.

31. The method of claim 27, wherein the at least one flexible strand is a suture strand and the at least one fixation device comprises two buttons.
